(19) United States
(12) Patent Application Publication
(10) Pub. No.: US 2008/0262294 A1
(43) Pub. Date: Oct. 23, 2008

(54) ENDOSCOPIC SYSTEM WITH DISPOSABLE SHEATH

(75) Inventors: Richard C. EWERS, Fullerton, CA (US); Eugene CHEN, Carlsbad, CA (US); Tung Thanh LE, Tustin, CA (US); Robert A. VAUGHAN, Laguna Niguel, CA (US); Marvin C. ELMER, Rancho Santa Margarita, CA (US); John A. COX, San Clemente, CA (US); Tracy D. MAAHS, Rancho Santa Margarita, CA (US)

Correspondence Address: LEVINE BAGADE HAN LLP, 2483 EAST BAYSHORE ROAD, SUITE 100, PALO ALTO, CA, 94303, US

(73) Assignee: USGI MEDICAL, INC., San Clemente, CA (US)

(21) Appl. No.: 11/738,297

(22) Filed: Apr. 20, 2007

(51) Int. Cl. A61B 1/005

(52) U.S. Cl. 600/104

(57) ABSTRACT

An endoscopic system includes a sheath having a flexible sheath body. A tip is attached to a distal end of the sheath body. A handle is attached to the proximal end of the sheath body. A steerable section may be provided in the sheath adjacent to the tip. Steering controls may then be provided on the handle for steering the steerable section. Lumens extend from the tip to the handle. The distal end of each lumen is sealed to the tip. Bodily fluids can only enter into the lumens and not other areas within the sheath. A shapelock assembly has an elongated hollow body positionable within the sheath body. The shapelock body may be switched between generally rigid and flexible conditions. The sheath provides a sterile barrier around the shapelock body. The shapelock assembly can be readily reused and the sheath may be disposable.

This application is related to U.S. patent application Ser. No. 11/238,298 filed Sep. 28, 2005 and now pending, which is a Continuation-in-Part of U.S. patent application Ser. No. 10/281,462 filed Oct. 25, 2002, now U.S. Pat. No. 6,690,163 B2, which is a Continuation-in-Part of U.S. patent application Ser. No. 10/173,227 filed Jun. 13, 2002, now U.S. Pat. No. 6,790,173, and a Continuation-in-Part of U.S. patent application Ser. No. 10/173,238 filed on Jun. 13, 2002, now U.S. Pat. No. 6,837,847, and a Continuation-in-Part of U.S. patent application Ser. No. 10/173,220 filed Jun. 13, 2002, now U.S. Pat. No. 6,783,491. The above listed applications are each incorporated herein by reference.

### BACKGROUND

Endoscopy is a minimally invasive medical procedure used to view areas inside of the body. By inserting an endoscope into the body, generally (but not necessarily) through a natural body opening, interior areas of the body may be viewed. Since endoscopic diagnoses or surgery do not require the types of large incisions that occur during conventional surgery, risks of complications are reduced and recovery tends to be relatively quick and painless in relation to conventional surgery.

The endoscope typically has a long thin flexible tubular body containing wiring and/or fiber optics to illuminate the viewing site and to transmit images of the viewing site to an eyepiece at the back end of the endoscope. The image may also be displayed on a video screen. The endoscope may also provide insufflation, irrigation, and/or suction. Endoscopic systems or tools are typically used with endoscopes to perform cutting, piercing, stitching, holding, etc. during endo-surgery.

Various endoscopic systems have been successfully used to perform a wide variety of diagnostic and surgical procedures. These include shapelocking tools, guides, or assemblies that provide advantages over conventional endoscopy or endosurgery. However, typical shapelocking assemblies include multiple highly engineered and precision manufactured components. As a result, they tend to be relatively more expensive and are intended to be reused many times. On the other hand, many components of endoscopic systems are well suited for single use only, because they are low cost or wear out too quickly to allow for safe and reliable reuse.

In addition, various endoscopic procedures are more effectively performed using endoscopic systems having varying features and capabilities. For example, some endoscopic procedures are more effectively performed using an endoscopic system having a shapelocking assembly, while other endoscopic procedures are more effectively performed using an endoscopic system having an elongated shaft that is flexible but not necessarily rigidizable. Other system capabilities, such as steerability, may be preferred for certain other procedures.

Accordingly, engineering challenges remain in designing endoscopic or endosurgical systems that achieve the advantages of using both reusable and disposable assemblies or components. In particular, these challenges remain in designing systems having modular components having various desirable functional capabilities that are capable of being exchanged, switched out, or otherwise selected by the user for use during a particular procedure.

### SUMMARY

In a first aspect, an endoscopic system has an elongated, tubular, flexible body member that is adapted to be used to perform diagnostic or therapeutic endoscopic procedures. The endoscopic system includes a first sub-assembly and a second sub-assembly that are combined to form the elongated, tubular, flexible body. In an embodiment, the first sub-assembly comprises a body member that provides structural support for the endoscopic system and the second sub-assembly comprises a sheath member that substantially maintains the first sub-assembly in a relatively clean or sterile condition. The endoscopic system also includes one or more control mechanisms configured to provide an interface for the user to operate the endoscopic system. In an embodiment, the one or more control mechanisms are provided on the first sub-assembly. In other embodiments, the one or more control mechanisms are provided on the second sub-assembly, or on both the first and second sub-assemblies.

In several embodiments, the endoscopic system includes a single use assembly and a reusable assembly. The single use assembly includes components adapted to act as a barrier against body fluids, to maintain the reusable assembly in a relatively clean or sterile condition. The single use assembly components are also adapted to support, position, hold, guide or steer endoscopic and endosurgical devices. The reusable assembly is configured to provide a shapelocking capability. The combination of the single use assembly and the reusable assembly provides versatile yet cost effective endoscopic systems.

### BRIEF DESCRIPTION OF THE DRAWINGS

In the drawings:

FIG. 1 is a front, top, and left side perspective view of an endoscopic system.

FIG. 2 is a top, back, and right side perspective view of the system shown in FIG. 1.

FIG. 3 is a front, top, and right side perspective view of the system shown in FIG. 1, during an intermediate assembly step.

FIG. 4 is an enlarged front, top, and right side perspective view of the handle portion of the system shown in FIG. 1.

FIG. 5 is an enlarged front, top, and right side perspective view of the system shown in FIG. 1, before complete assembly.

FIG. 6A is a front, top, and right side perspective view of the handle shown in FIGS. 4 and 5, and illustrating the hinge connecting the handle barrel to the handle base.

FIGS. 6B-C are front and back perspective views, respectively, of another embodiment of a handle portion of an endoscopic system.

FIG. 7A is a front, top, and left side perspective view of a first embodiment of a shapelock assembly.

FIG. 7B is a front, top, and left side perspective view of a second embodiment of a shapelock assembly.

FIG. 8A is an enlarged front, top, and left side perspective view of the back end of the shapelock assembly shown in FIG. 7A.

FIG. 8B is an enlarged front, top, and left side perspective view of the back end of the shapelock assembly shown in FIG. 7B.

FIG. 9 is a schematically illustrated side view of a sheath portion of the endoscopic system shown in FIG. 1.

FIG. 10 is an enlarged schematically illustrated side view of the sheath tip shown in FIG. 9.

FIG. 11 is a schematically illustrated section view taken along line 11-11 of FIG. 9.

FIG. 12 is a schematically illustrated side view diagramming a first step in manufacturing the sheath shown in FIG. 9.

FIG. 13 is a schematically illustrated side view showing a second step in manufacturing the sheath shown in FIG. 9.

FIG. 14 is a schematically illustrated side view showing a subsequent step in the manufacture of the sheath shown in FIG. 9.

### DETAILED DESCRIPTION OF THE DRAWINGS

As used herein, the term “endoscopic” means of, relating to, or performed by means of and endoscope or endoscopy, and includes viewing, manipulating, treating, or performing surgery on or at a site inside of the body, without opening up the body or only minimally opening up the body. Hence, as used herein, endoscopic includes endosurgical, endoluminal, laparoscopic, and the like. As used herein, the term “shapelock assembly” means an apparatus that may be transitional or switched (in whole or in part) between a generally flexible condition and a generally rigid condition. Several examples of shapelock assemblies are described, for example, in U.S. Pat. Nos. 6,783,491 and 6,960,163, and United States Patent Application Publication No. US 2006/005852, each of which is incorporated by reference herein.

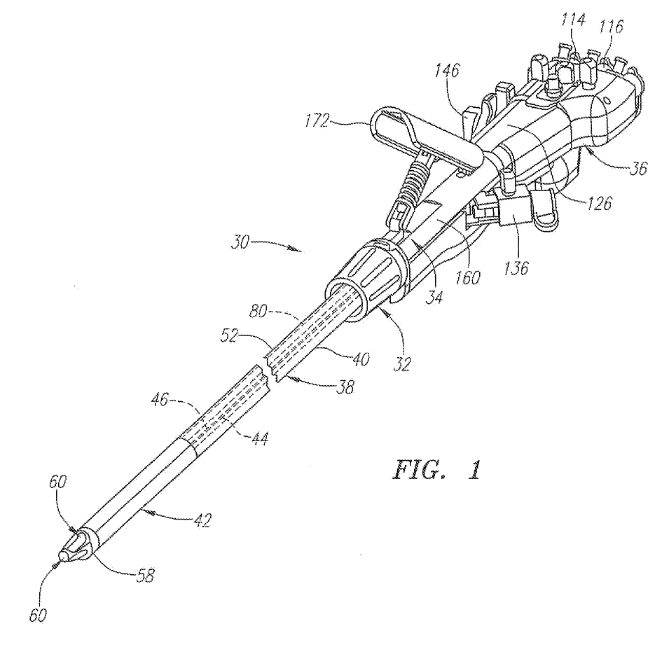

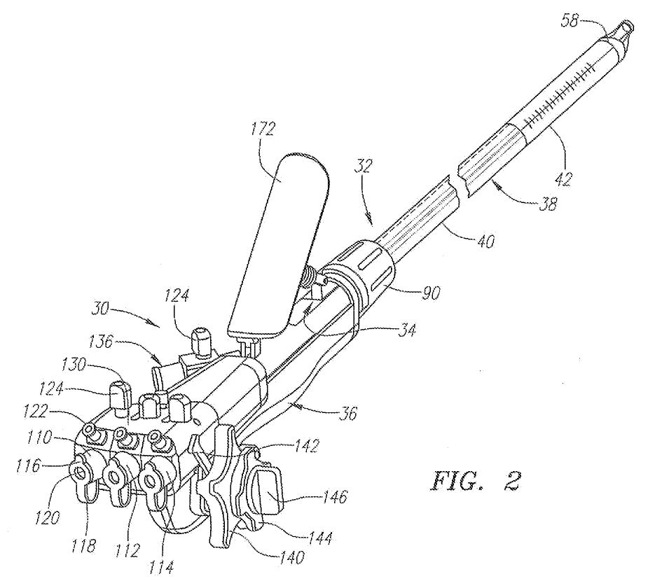

Turning to the drawings, as shown in FIGS. 1 and 2, an embodiment of an endoscopic system 30 includes a reusable shapelock assembly 34 that is adapted for use within a disposable assembly generally designated 32. The disposable assembly 32 has a flexible sheath 38 attached to a relatively rigid handle 36.

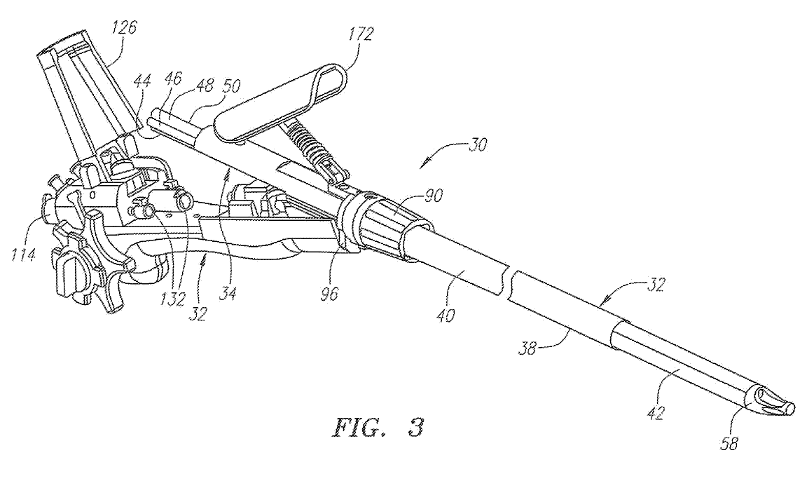

Referring also to FIGS. 3, 4, and 5, the sheath 38 includes a tip 58 attached to the distal end of a steerable section 42. A body section 40 of the sheath 38 extends proximally from the steerable section 42 to the handle 36. A lock nut 90 or similar attachment holds the proximal end of the body section 40 of the sheath 38 onto the handle 36.

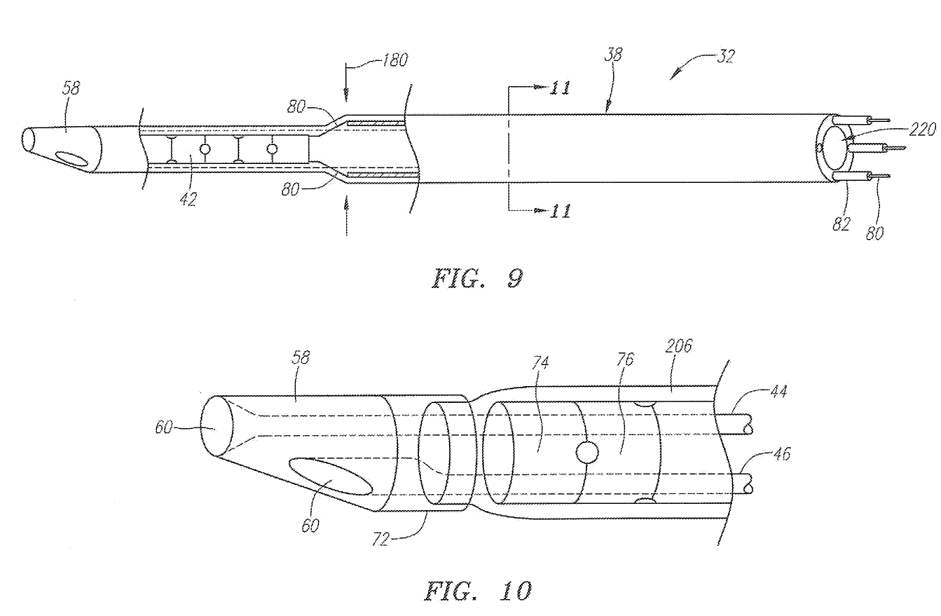

Referring to FIGS. 9 and 10, the sheath tip 58 has one or more tool lumen openings 60. A plurality of tool lumens extend through the interior of the sheath 38. The tool lumens are defined by one or more structural guideway members provided within the interior of the sheath 38. In the embodiments shown, the tool lumens are defined by a plurality of sleeves or tubes 44, 46, 48, and 50, each of which has an open distal end that is sealed around a respective lumen opening 60 in the tip 58. The tubes 44, 46, 48, and 50 extend proximally from the tip 58 through the length of the sheath 38 to the handle 36. In an embodiment, the tubes 44-50 are flexible rubber or plastic tubes that act as guideways between the handle 36 and the tip 58 for tools and instruments, as described below. In other embodiments, the tubes 44-50 are flexible tubes having composite construction, such as a multi-layer extrusion, or coil and/or braid reinforced construction. In some embodiments, the tubes 44-50 are constructed to reduce or eliminate the likelihood that the tubes will become twisted, kinked, tangled, torn, or that the lumens defined by the tubes would collapse under vacuum.

The embodiments described above and illustrated in the Figures include a plurality of tubes that serve as structural guideway members used to define the tool lumens extending through the sheath 38. In other embodiments, the tool lumens are defined by other structures that serve as guideway members. For example, in some embodiments, the tool lumens are defined by one or more partition members extending partially or completely through the sheath 38. In an embodiment, the partition members define tool lumens having a circular cross-section, while in other embodiments the partition members define lumens having non-circular (e.g., triangular, rectangular, square, oval, irregular, or other) cross-sectional shapes. In an embodiment, the partition members are removably attached to the interior of the sheath. In other embodiments, the partition members are formed integrally (e.g., co-extruded) with the sheath.

Referring still to FIGS. 9 and 10, an embodiment of the steerable section 42 at the distal end of the sheath 38 is formed by segments, links, or other generally rigid and pivotably interconnected elements. In the example shown in FIGS. 9 and 10, the steerable section 42 includes links, with the first link shown at 74 and the second link at 76. The length and flexibility of the steerable section 42 is varied by selecting the number and size of the individual links making up the steerable section 42. Steering wires 80 or other steering elements are typically attached to the first link 74, for steering the steerable section 42. The steering wires 80 extend proximally from the first link 74, through the second link 76 and any additional links of the steering section 42, and through the sheath 38 to the handle 36. In an embodiment, the steering wires 80 are provided within coils 82 or other column strength element. The coils 82 allow the steering wires 80 to be tensioned without buckling the sheath 38. The steering section generally is about 4-10 cm long, whereas the sheath 38 is typically between 20-200 cm. The sheath outer diameter is typically 1-2.5 cm.

While FIGS. 9 and 10 show the steerable section 42 operated via steering wires 80, other forms of steering elements may be used, including elements acting in tension, such as steering wires, or elements acting in compression, such as push rods. The steering section 42 may alternatively be controlled electrically, pneumatically, or hydraulically. The specific technique used for controlling the steerable section 42 may vary.

Referring to FIG. 10, the sheath 38 has an outer skin or layer 206 formed of a material that is flexible and that provides a protective layer to prevent passage of bodily fluids, gases, and other materials, such as a polymeric or plastic material or the like. In an embodiment, the cylindrical proximal end 72 of the tip 58 is sealed onto the outer skin 206 via an adhesive or other bonding or attachment method. In other embodiments, the tip 58 is removably attached to the distal end of the sheath 38, such as by screwing, friction fit, or other mechanism adapted to provide the user with the ability to exchange tips for various procedures. In an embodiment, the tip 58 is made of hard or soft plastic or rubber, or similar non-porous materials. Because the sleeves or tubes 44-50 are sealed at the lumen openings 60 of the tip 58, gases and liquids encountered during use of the system 30 within the body are substantially prevented from entering into the sheath 38, except through the tubes 44-50. Accordingly, the internal spaces within the sheath 38 are not exposed to liquids or gases in use.

As noted above, in the embodiments described, the tip 58 is formed of hard or soft plastic or rubber, or similar non-porous materials. In an embodiment, the tip 58 is formed of a transparent plastic, rubber, or polymeric material. In an embodiment, the tip 58 is substantially dome-shaped, and the openings 60 are substantially equally distributed over the tip 58. In the embodiments shown in the Figures (see, e.g., FIG. 10), the tip 58 include an asymmetrical, conical shape in which the openings 60 for the tool lumens are not equally distributed. For example, the opening 60 for one of the tool lumens is located at a point on the tip 58 that is distal of the other openings 60. This orientation provides the ability to have endoscopic tool exit ports that are longitudinally staggered. For example, in an embodiment, the opening 60 for an endoscope tool lumen is located distally of the opening 60 for a second diagnostic or therapeutic endoscopic tool. In this way, the second tool is able to be observed as it exits the tip 58, rather than after it has extended substantially from the tip 58. Other variations are also possible.

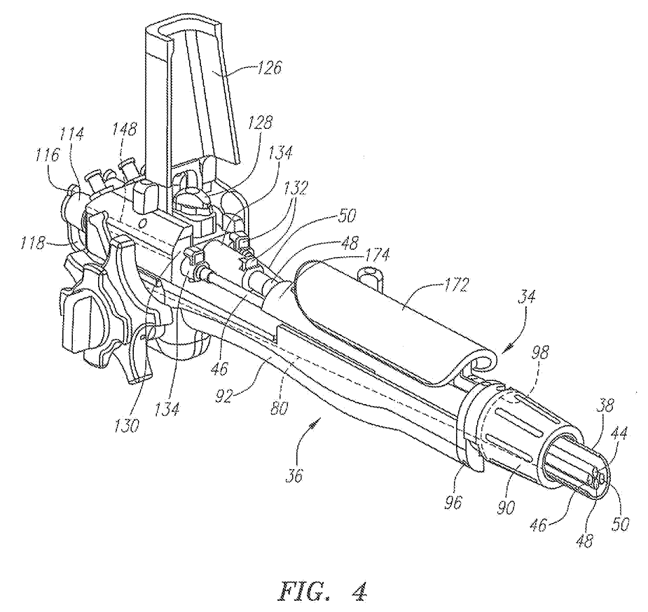

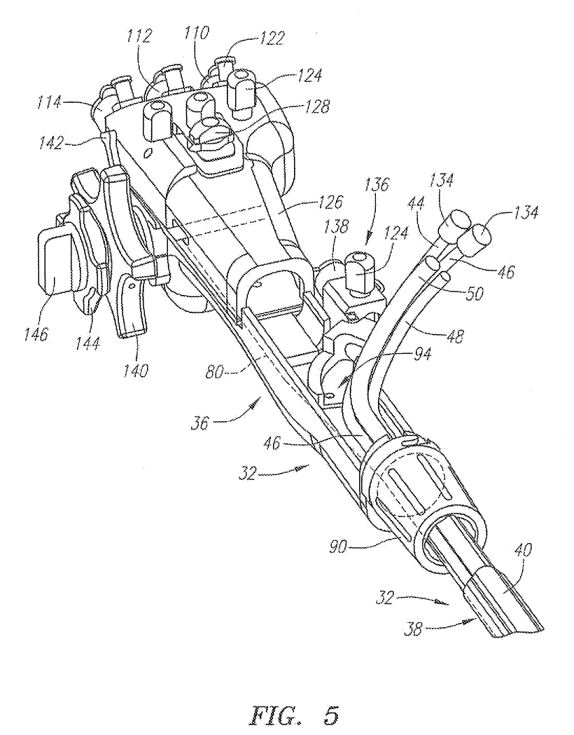

Referring once again to FIGS. 4, 5, and 10, the tubes 44-50 extend proximally within the sheath 38 from the tip 58 to the handle 36. As shown in FIG. 5, the proximal ends of the tubes 44-50 are initially free, i.e., unattached to the handle 36. The tubes 44-50 each have a lumen end fitting 134 used to attach the proximal ends of the tubes to a lumen block 130 on the handle 36.

In an embodiment, the steering wires 80 and coils 82, or other tension or compression steering elements used, extend proximally through the sheath 38 to steering control knobs 140 and 144, as shown in FIG. 5. In the illustrated embodiments, four steering wires or elements 80 are used. The first and third wires, oriented on opposite sides of the first link 74, are connected on a shaft or spool joined to the first control knob 140, for example, to provide vertical or up/down steering movement to the steerable section 42. Similarly, second and fourth steering wires or elements 80 are attached to a shaft or spool joined to the second steering control 144, to provide lateral or left/right steering movement to the steerable section 42. In the illustrated embodiment, steering brake or locking controls 142 and 146 are associated with each steering control 140 and 144.

In the embodiments shown, the sheath 38, which includes or contains the tip 58, steering section 42, tubes 44-50 and steering elements, is attached to the handle 36 during manufacture.

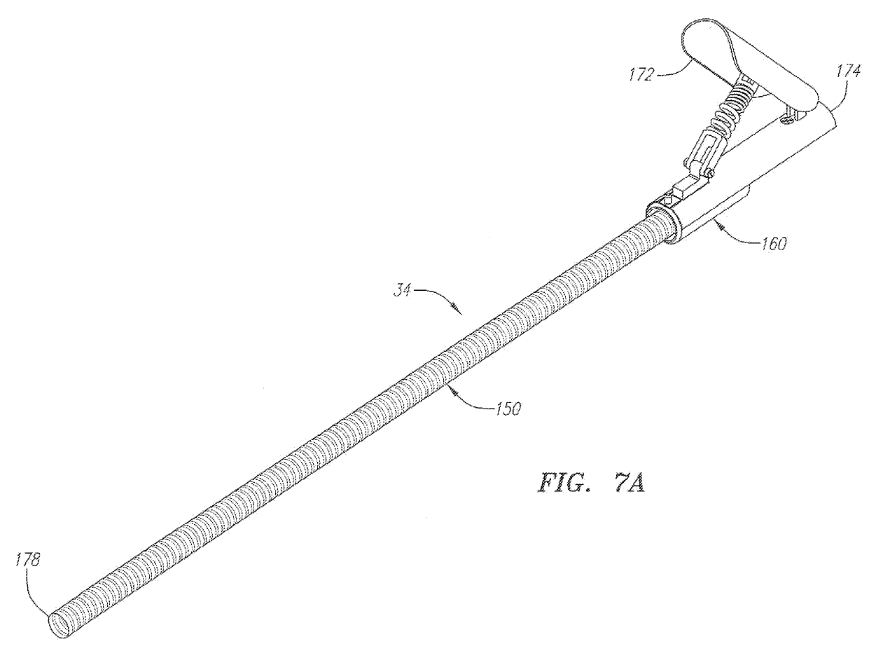

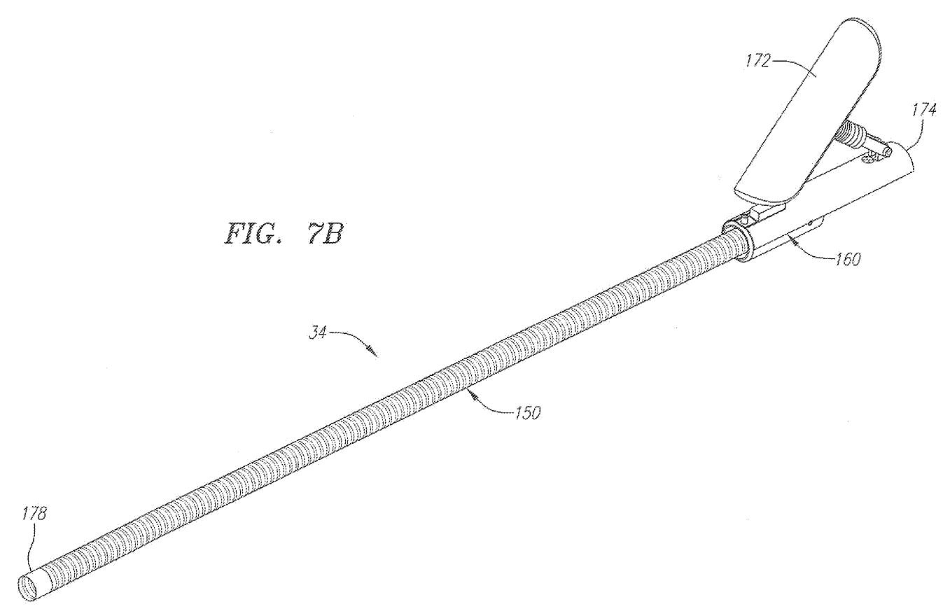

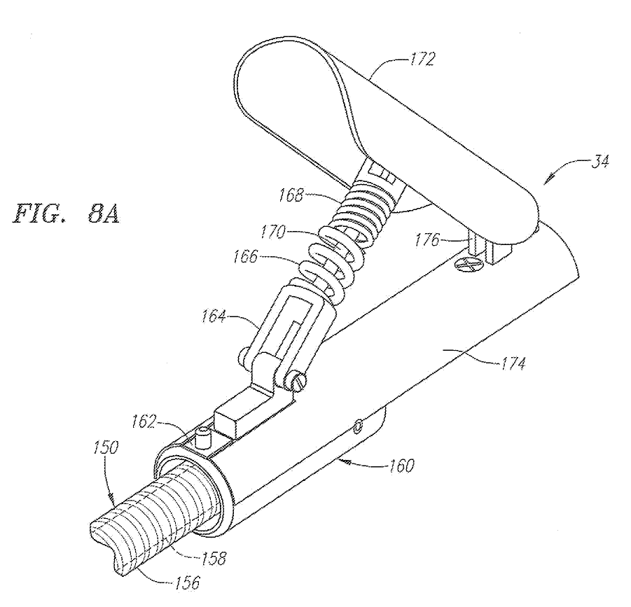

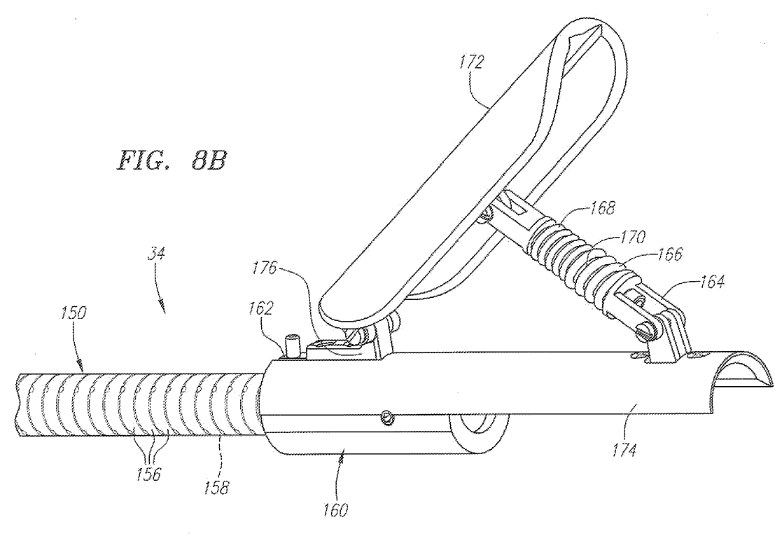

FIGS. 7A-B and 8A-B show two embodiments of a shapelock assembly 34. A first embodiment, shown in FIGS. 7A and 8A, includes a locking handle 172 that opens at its distal end, whereas the second embodiment (FIGS. 7B and 8B) includes a locking handle 172 that opens at is proximal end. With the exception of the locking handle orientation, the illustrated embodiments are functionally identical. In some embodiments, the shapelock assembly 34 is reusable. In the examples shown in FIGS. 7A-B and 8A-B, the shape lock body 150 is formed by segments, such as links 156 that are pivotably attached to (or positioned next to) each other in a nested arrangement. In an embodiment, the links 156 are formed as nested rings, so that the shapelock body 150 is tubular, or has an open internal through passageway. Lock wires 158 extend from a base 160 to the distal end 178 of the shapelock body 150.

As shown in FIGS. 8A-B, the lock wires 158 pass through a compression collar 162 in the base 160. In the FIG. 8A embodiment, the proximal end of the locking handle 172 is supported on a handle pivot block 176 on a base extension 174. In the FIG. 8B embodiment, the distal end of the locking handle 172 is supported on the handle pivot block 176. A spring shaft 170 is pivotably attached to the locking handle 172 at a position spaced apart from the pivot block 176. Springs 166 and 168 are supported on a clevis 164 and positioned around the spring shaft 170. Pushing or squeezing the locking handle 172 down toward the base 160 causes the compression collar 162 to compress the nested links 156 against one another over the fixed-length lock wires 158. This causes the shape lock body 150 to become substantially rigid. With the handle in the open position as shown in FIGS. 7A-B and 8A-B, compression of the links 156 on the lock wires 158 is released, and the shape lock body 150 is generally flexible. The first spring 166 has a lower spring constant than the second spring 168. This allows for easier initial downward pivoting movement of the lock handle 172, with relatively lower force. As the locking handle 172 moves down toward the base 160, where the user's hand has greater mechanical advantage, the second stiffer spring 168 applies proportionally increasing force to the compression collar 162. In an alternative embodiment, a single spring is used and the biasing force is provided by only the single spring through the entire travel of the locking handle 172.

The shapelock assembly 34 embodiments shown in FIGS. 7A-B and 8A-B are examples of various shapelocking assemblies that are suitable for use in the endoscopic system 30. The specific shapelock assembly mechanism used is not essential, so that a large number of alternative mechanisms that can transition from substantially flexible to substantially rigid may be used. Examples of alternative shapelock assembly mechanisms suitable for use in the endoscopic system are described, for example, in U.S. Pat. Nos. 6,783,491 and 6,960,163, and United States Patent Application Publication No. US 2006/005852, each of which is incorporated by reference herein. These patents and publications also provide additional details about the structure and operation of the shapelocking assemblies described herein.

In an embodiment, the links 156 and other elements forming the shapelock body 150 advantageously have high strength and yet are light weight. The locking mechanism associated with the shapelock body typically is also advantageously highly reliable. In some embodiments, the shapelock assembly 34 portion of the endoscopic system 30 is relatively more costly to construct (in terms of labor, time, and/or expense) than is the disposable assembly 32. Moreover, in some embodiments, it is relatively more simple, efficient, and/or cost-effective to clean and/or re-sterilize the shapelock assembly 34 portion of the endoscopic system 30 alone or independently of the disposable assembly 32. For example, in some embodiments, the disposable assembly 32 includes rubber coatings, steering links, lumens, and/or other components that are less easily, efficiently, or effectively cleaned and/or sterilized. Accordingly, there are significant advantages in being able to reuse the shapelock assembly 34 over multiple procedures and/or with multiple patients, and to dispose of the disposable assembly 32 after a single or limited number of patient uses.

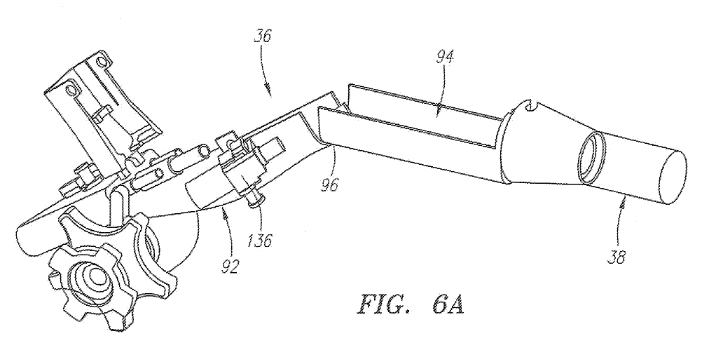

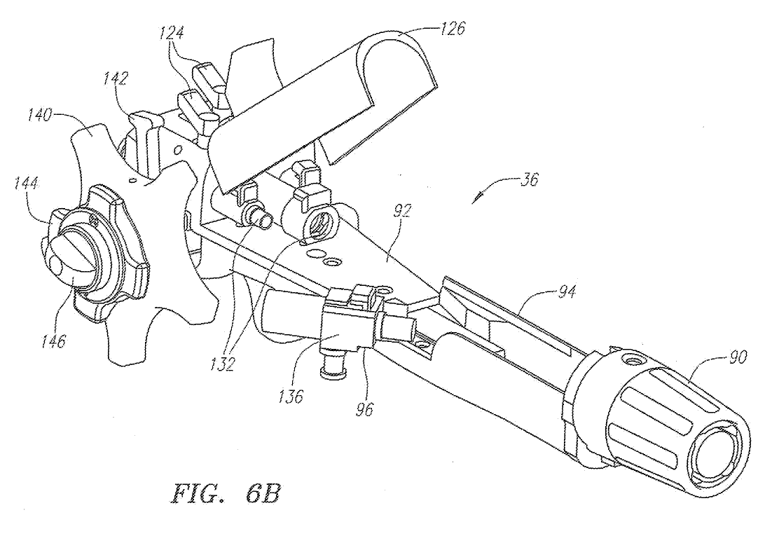

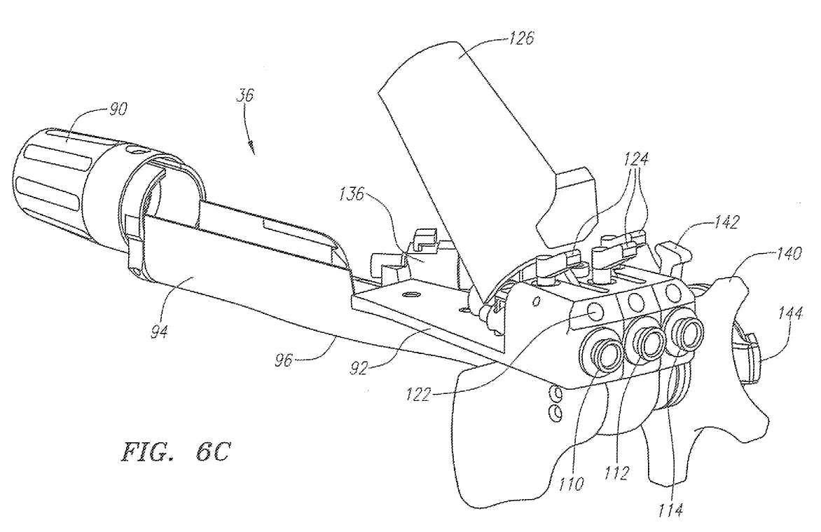

In an embodiment, the disposable assembly 32, which includes the handle 36, and sheath 38, is provided in a sterile condition, for example, within a sterile package. To prepare for use, e.g., in an operating room, the assembly 32 is removed from the sterile package, in the form as shown in FIG. 5. The sleeves or tubes 44-50 are loose or unattached at their proximal ends. The handle barrel 94 is pivoted downwardly relative to the handle base 92 about the hinge 96, as shown in the embodiment illustrated in FIG. 6A. FIGS. 6B-C show another embodiment of the handle 36 in its locked position, without being pivoted about the hinge 96. The shapelock assembly 34 is then installed into the disposable assembly 32 as follows. The shapelock assembly 34 is removed from its sterile package, if any. With both the handle/sheath assembly 32 and the shapelock assembly 34 in a sterile field (for example on a sterile sheet on an operating room cart or table), the proximal ends of the tubes 44-50 are threaded into the open distal end 178 of the shapelock assembly 34. The shapelock assembly 34 may be in a rigid or in a flexible condition. The body 150 of the shapelock assembly is advanced into the sheath 38 until the distal end 178 of the shapelock body 150 is adjacent to the steerable section 42, at position 180 shown in FIG. 9. Once the shapelock assembly 34 is moved into this position, the tubes 44-50 extend out from the proximal end of the shape lock body 150, beneath the base extension 174, as shown in FIG. 3.

During installation of the shapelock assembly 34 into the disposable assembly 32, the cover 126 on the handle 36 is in the up or open position. The proximal end of each tube 44-50 is then connected to a respective fitting 132 on the lumen block 130 on the handle 36. Referring to FIG. 4, the tube fittings 134 on the tubes are clamped in place via lumen clamps 124. Lumen bores 148 extend from the distal end of the lumen block 130, where the tubes 44-50 are attached, to lumen entry guides 110, 112, and 114 on the proximal end of the handle 36, as shown in FIG. 2. A tether 118 holds a cap 116 containing a cap seal 120 on each of the lumen entry guides 110-114. A Luer port 122 connects into each lumen bore in the lumen block 130, distally of the lumen entry guides 110-114.

With the shapelock assembly 34 installed and the tubes 44-50 connected to the handle 36, the handle barrel 94 is pivoted back to the closed position as shown in FIG. 4. The cover 126 is closed and may be locked by turning a cover lock knob 128, as shown in FIGS. 2 and 5. The flat bottom side of the base extension 174 of the shapelock assembly 34 is supported on a flat raised surface in the handle 36, to keep the shapelock assembly 34 stabilized relative to the handle 36.

One or more endoscopic tools may be inserted into and through the system 30 via the entry guides 110-114, with the distal ends of the tools moved through the sleeves or tubes 44-50 to the tip 58. In the embodiment shown, the cap seals 120 seal against the shafts of the tools. This prevents extensive leaking of insufflation air or gas. In other embodiments, seals are provided at other locations along the tool lumens, such as within the handle 36, at an intermediate portion in the sheath 38, and/or at or near the distal ends of the tool lumens near the tip 58. In an embodiment, thumb screws 124 on the handle 36 are tightened to prevent sliding front/back movement of the tools within the lumens. In an embodiment, one or more of the Luer ports 122 are connected to a gas/insufflation source, a liquid source, or a vacuum source. Referring to FIGS. 2 and 5, the proximal ends of the tubes 44-50 in the handle 36, and the lumen bores in the lumen block 130 are substantially straight. This allows endoscopic tools to be moved into the system 30 easily, since the endoscopic tools move in a substantially straight path when installed into the system 30.

Referring to FIGS. 1 and 5, in an embodiment, a side access fitting 136 is provided on the side of the handle 36, with one of the tubes 44-50 connecting into a side lumen entry guide 138. The side access fitting 136 allows one or more endoscopic tools to be positioned off to the side of the handle 36, away from the position of the other endoscopic tools extending out from the back of the handle. The side access fitting 136 is preferably oriented at an angle of 5-90 degrees or more, and more preferably 15-60 degrees, relative to the longitudinal axis of the sheath 38. This allows one user to use, e.g., an endoscope routed through the side access fitting 136, while a second user operates other tools routed through the other lumens, with each user having more room around the handle 36.

The tip 58 and sheath 38 are then moved into the patient. For diagnostic, therapeutic, or surgical procedures in the gastrointestinal (GI) tract, the sheath 38 is inserted into the mouth and throat and guided through the esophagus to the stomach or other location. For procedures involving the large intestine or colon, the sheath is guided through the anus and rectum. In an embodiment, a hydrophilic coating is applied to the outside surface of the sheath 38 to allow the sheath to slide more easily through body openings. In other embodiments, the sheath 38 is provided with a coating that includes one or more of a bactericide, a pain reducer, a relaxant, and/or another facilitating or therapeutic material suitable for use as a coating over the sheath.

To maneuver within these or other hollow body organs or to position the tip 58 for a specific procedure, the steering controls 140 and 144 are used to steer the steerable section 42. To better hold the sheath 38 into a desired position or shape, the shapelock assembly 34 may be made rigid by pressing the lock handle 172 down, from the position shown in FIGS. 1 and 2, to the position shown in FIG. 4. This places the shapelock assembly 34 into a substantially rigid condition, which correspondingly locks the sheath 38 surrounding the shape lock 34 into the same shape or position.

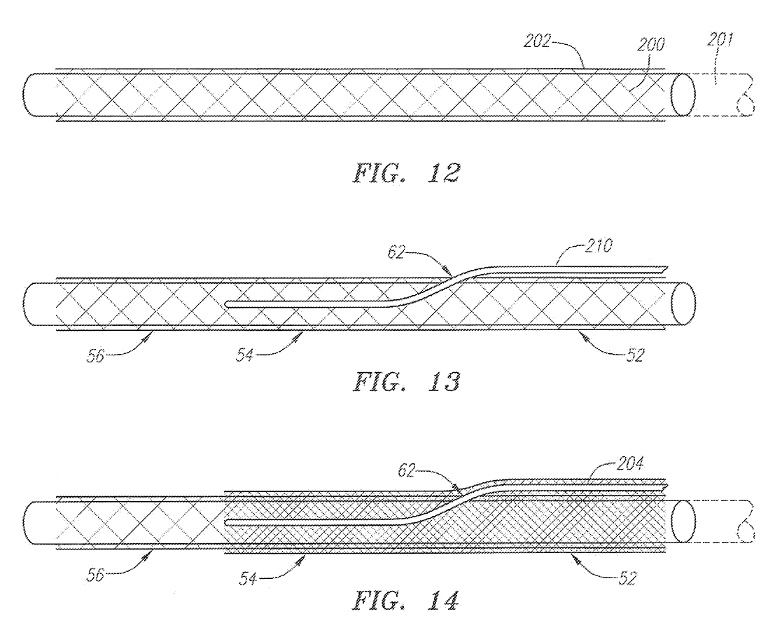

FIGS. 12-14 show an example of a method of manufacturing a sheath 38 suitable for use in the disposable assembly 32 embodiments described herein. A first or inner layer 200 of a braid or woven mesh of a reinforcement material is placed onto a mandrel 201, as shown in FIG. 12. In an embodiment, the mandrel 201 is a cylindrical rod (e.g., aluminum) having a thin polymeric (e.g., polyolefin shrink tube) coating. In an embodiment, the reinforcement material is a mesh formed of a polymeric material such as polyethylene terephthalate (PET) or other suitable reinforcement material. A second layer 202 of reinforcement material is then applied over the first layer 200. The second layer 202 of reinforcement material will extend over the entire length of the sheath 38, so a sufficient length of the material is placed on the mandrel 201. The first or inner layer 200 of reinforcement material is shorter than the second layer 202, extending from the proximal end of the sheath 38 and terminating at the steerable section 42. Passageway forming wires 210 are then temporarily installed. Each wire 210 will form a passageway in the sheath for a steering coil and wire. The forming wires have a diameter nominally larger than the steering coils 82. If the sheath will have four steering wires, then four forming wires 210 are installed, equally radially spaced apart (i.e., at 90 degree intervals around the circumference). The passageway forming wires 210 are installed between the first layer 200 and the second layer 202 of reinforcement material, and are installed only in the section of the sheath 38 that the first layer 200 and second layer 202 overlap. In an embodiment, the forming wires 210 are stainless steel wires coated with polytetrafluoroethylene to facilitate removal of the wires from the sheath after the skin layer 204 is formed, as described below.

In other embodiments, the order of installation of the first layer 200 of reinforcement material, the second layer 202 of reinforcement material, and the passageway forming wires 210 are different. For example, in an embodiment, the passageway forming wires 210 are installed after the first layer 200 is installed, but before the second layer 202 is installed. A person of ordinary skill in the art will recognize that other orderings are also possible.

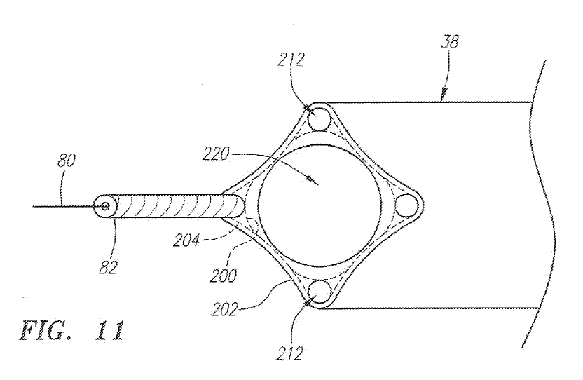

Once the first layer 200 of reinforcement material, second layer 202 of reinforcement material, and passageway forming wires 210 are installed as described above, a skin layer 204 is applied over the first and second layers. The skin layer 204 is formed of a flexible material that is capable of providing the performance capabilities desired for the sheath 38. In an embodiment, the skin layer 204 is formed of a polymeric material such as plastisol that is applied using a dipping and/or spinning process. The skin layer 204 is applied over the section of overlap of the first layer 200 and second layer 202, and over the remainder of the second layer 202 that is not overlapping the first layer 200. After drying or curing the skin layer 204, the forming wires 210 are pulled out or otherwise removed. This leaves passageways 212 for the steering wires within the walls of the sheath 38, as shown in FIG. 11. Specifically, each passageway 212 is located within the skin material and in between the first and second layers of braid or reinforcement material. In an embodiment, the passageways 212 are formed in straight, radially spaced alignment extending over the proximal length of the sheath 38. In other embodiments, the passageways 212 include partial spiral sections that are provided in order to locate the proximal and distal exit points of the steering wires 80 from the sheath 38 as desired.

Referring to FIGS. 9 and 11 at position 180, the steering wires 80 exit from the passageways 212, run inside of the steerable section 42, and attach to the front segment of the steerable section 42. Referring to FIG. 9, distally of position 180, the sheath includes a single layer of reinforcement and skin. Toward the proximal end of the sheath 38, the steering wires are contained within the passageways 212 and extend to the handle 36, where the steering wires 80 are operably connected to the control knobs 140 and 144. Consequently, the shape lock 34 may be inserted into the central sheath opening 220 without contacting or interfering with the steering wires.

In other embodiments, the layers of reinforcement material 200 and 202 are tubes of metal or plastic with a braided or woven structure. More or fewer reinforcement layers may be included over either a portion or the full length of the sheath 38 in order to change the stiffness, torque transmission capabilities, strength, or other material properties of the sheath 38. In other embodiments, various materials may be used for the skin, including other polymers applied by dipping, spinning, or extruding processes. Since the reinforcement materials will generally have an open structure, some skin material may pass through it to the mandrel, tending to create a smooth inside wall surface. In other embodiments, the reinforcement materials substantially prevent the skin material from penetrating, such that the first layer 200 of reinforcement material is partially or fully exposed on the interior surface of the sheath 38. The materials, construction, and design details of the sheath may be varied, provided that the sheath is substantially flexible and substantially prevents the shapelock from coming into contact with body fluids.

Although the endoscopic system embodiments described above include a reusable portion that includes a shapelock assembly, other embodiments include other components. For example, in some embodiments the sheath has sufficient strength and stiffness to allow it to be used without a shapelock assembly. In those embodiments, a non-shapelocking core or skeleton member (either rigid or flexible), a stiffening spring, a coil, a tube, or a rod is placed into the central opening 220 of the sheath, instead of the shapelock assembly. In other embodiments, the sheath and handle assembly are used with no additional support component. In addition, although the embodiments described include a plurality of guideway members defining tool lumens, in other embodiments the guideway members define only a single lumen, or two or more lumens.

Thus, novel methods and apparatus have been shown and described. Various changes and substitutions may of course be made without departing form the spirit and scope of the invention. The invention, therefore, should not be limited except by the following claims and their equivalents.

1. An endoscopic system, comprising:
a sheath having a flexible sheath body, a tip at a first end of the body, and a handle at a second end of the body, a steerable section adjacent to the tip, steering controls on the handle for steering the steerable section, and two or more lumens extending from the tip to the handle; and
a shapelock assembly having a shapelock body positionable within the sheath body, and with the sheath providing a barrier around the shape lock.

2. An endoscopic system, comprising:
a first assembly including:
a handle;
a flexible sheath including:
a tip;
a steerable section; and
a body section;
with the body section attached to the handle, and with the steering section attached to the body section, and with the tip attached to the steerable section;
at least one tool lumen extending from the tip, through the steerable section and the body section to the handle;
pull or push elements in the sheath extending from the steerable section through the body section and connecting to a steering control in the handle; and
a second assembly including:
a plurality of adjacent elements forming a tube;
a locking system associated with the adjacent elements for locking the adjacent elements to make the tube substantially rigid, and for releasing the adjacent elements to make the tube flexible;
with the tube removably insertable into the sheath, and with the tool lumens extending through the tube.

3. The system of claim 2 with the handle including a barrel section pivotally attached to a back section.

4. The system of claim 3 with the steering control on the back section.

5. The system of claim 2 with the sheath including a first layer of material and a second layer of material, and with the push, or pull elements extending through tubes positioned between the first and second layers of material.

6. The system of claim 5 with the push or pull elements comprising at least two steering wires each extending through a tube in the sheath.

7. The system of claim 2 wherein the tip is substantially optically transparent.

8. The system of claim 2 with the sheath comprising an outer rubber or plastic skin.

9. The system of claim 8 further comprising a hydrophilic coating on the skin and/or in the lumens.

10. The system of claim 3 with the back section of the handle including lumen ducts passing through a lumen block, and with each of the lumen ducts within 20 degrees of parallel to a central axis of the handle.

11. An endoscopic system, comprising:
a handle;
a flexible sheath including:
a tip;
a steerable section; and
a body section;
with the body section attached to the handle, and with the steering section attached to the body section, and with the tip attached to the steerable section;
at least one tool lumen extending from the tip, through the steerable section and the body section to the handle;
a tool lumen seal sealing the tool lumen to the tip, to prevent entry of fluids into the sheath;
steering elements extending from the steerable section through the body section and connecting to a steering control in the handle; and
a flexible elongated tubular stiffener within the sheath, and with the sheath forming a barrier around the stiffener.

12. The endoscopic system of claim 12 with the stiffener comprising a coil member.

13. The endoscopic system of claim 12 with the stiffener comprising a shapelock assembly.

14. An endoscopic system, comprising:
a sheath assembly including:
a flexible sheath attached to a handle, with the sheath including:
a tip;
a steerable section; and
a body section;
with the body section attached to the handle, and with the steering section attached to the body section, and with the tip attached to the steerable section;
at least one tool lumen extending from the tip, through the steerable section and the body section to the handle;
a tool lumen seal sealing the tool lumen to the tip, to prevent entry of fluids into the sheath;
steering wires in the sheath extending from the steerable section through the body section and connecting to a steering control on the handle; and
a shapelock member within the sheath, and with the tool lumens within the shapelock member, and with the steering wires outside of the shapelock member.

15. The system of claim 14 with the steering wires located outside of the steerable section and extending into passageways formed in body section of the sheath.

16. The system of claim 14 further comprising at least one lumen bore in the handle, and with the tool lumen connectable to the lumen bore.

17. The system of claim 16 further comprising a side access fitting on the handle having an access channel oriented at an angle of 15-60 degrees to the tool lumen.

18. An endoscopic method comprising:
installing a shapelock body of a shapelock assembly within a central opening of a sheath,
moving the sheath containing the shapelock body into an orifice of a patient;
moving an endoscopic instrument through a tool lumen to a site within the patient, with the tool lumen within the shapelock body and within the sheath;
actuating the shapelock assembly to make the shapelock body substantially rigid; and
with the sheath substantially preventing fluids in the body of the patient from contacting the shapelock body.

19. The method of claim 18 further comprising steering a tip of the sheath.
